(19) United States
(12) Patent Application Publication
(10) Pub. No.: US 2017/0079903 A1
(43) Pub. Date: Mar. 23, 2017

(54) SKIN COSMETIC

(71) Applicant: Henkel AG & Co. KGaA, Duesseldorf (DE)

(72) Inventors: Tanja Boesel, Langenfeld (DE); Annika Baehren, Juechen (DE); Marianne Waldmann-Laue, Monheim (DE)

(73) Assignee: Henkel AG & Co. KGaA, Duesseldorf (DE)

(21) Appl. No.: 15/257,623

(22) Filed: Sep. 6, 2016

(30) Foreign Application Priority Data
Sep. 22, 2015 (DE) 10 2015 218 204.5

(51) Int. Cl. A61K 8/97; A61K 8/55; A61Q 19/00; A61K 8/60

(52) U.S. Cl. CPC A61K 8/97; A61K 8/602; A61K 8/55; A61Q 19/00; A61K 2800/5922

(57) ABSTRACT

An aqueous cosmetic composition includes:
a) at least one alkyl glucoside including a linear or branched alkyl or alkenyl group having 14 to 30 carbon atoms and a mono-, di- or triglucoside group;
b) at least one extract of a plant species from the aralia family (Araliaceae);
c) at least one extract of a plant species from the pine family (Pinaceae);
d) at least one extract of a plant species from the flowering plants family (Theaceae); and
e) at least one extract of a plant species from the gooseberry family (Grossulariaceae).

### FIELD OF THE INVENTION

The present invention generally relates to cosmetic compositions for caring for skin.

### BACKGROUND OF THE INVENTION

As the largest organ of humans, the human skin performs a variety of vital functions. Having, on average, a surface area of approximately 2 m² in human adults, the skin plays a major role as a protective and sensory organ. Tasks of this organ include conveying and warding off mechanical, thermal, actinic, chemical and biological stimuli. The skin, for example, also protects the human organism from the damaging effects of a wide variety of microorganisms, and yet, despite protective mechanisms of its own, is possibly also damaged itself by these microorganisms.

Inflammatory conditions of the skin are caused by harmful bacteria, such as Propionibacterium acnes, which are permanently present on the skin, but proliferate more drastically under certain conditions and cause “blemished skin” or acne, for example. These are essentially microorganisms (bacteria and fungi) that must be classified as pathogenic.

At the same time, the resident bacterial skin flora is also composed of other bacterial species; however, these do not cause damage, but the growth thereof rather keeps harmful microbes in check and, as a result, these perform an important protective function. These include above all coagulase-negative staphylococci such as S. epidermidis. In general, harmless, desirable skin microbes are also referred to by the term “saprophytic.” However, there are also desirable skin microbes that, according to the present classification, are not assigned to the group of “saprophytic” skin microbes, just as there are microbes classified as being “pathogenic” which may be desirable under certain circumstances, so that a distinction between “desirable” and “undesirable” does not equate to a distinction between “saprophytic” and “pathogenic.” In this regard, it must in particular also be considered that harmless skin-friendly microbes can become pathogenic with excessive reproduction. The classification of bacteria as pathogenic is thus possibly dependent on the composition of the microbial skin flora.

Non-selective antibacterial active ingredients, such as are used for preventing and controlling acne in commercially available cosmetics, for example, not only kill undesirable skin microbes, but also desirable skin microbes and thereby disrupt the biological equilibrium, which can have a variety of undesirable consequences.

A need therefore exists for agents that selectively bring about an advantageous microbiological equilibrium at the application site, this being the skin, for example by promoting the growth and/or the viability of desirable microbes of the skin flora over the growth and/or the viability of undesirable microbes of the skin flora. Corresponding substances are also referred to as “prebiotic.”

So far, the detection of a prebiotic effect of substances has essentially been limited to the intestine. Various publications, for example, describe the use of substances that promote the growth of desirable intestinal bacteria.

The use of prebiotic plant extracts in skin cosmetics is described in the German patent application DE 10333245 A1. This application considers the group of prebiotic plant extracts to include, among other things, extracts of ginseng (Panax ginseng) and black currant (Ribes nigrum).

German patent application DE 102012223405 A1 describes a skin care agent that, in addition to other components, includes extracts of ginseng (Panax ginseng), Scots pine (Pinus sylvestris), tea (Camellia sinensis) and black currant (Ribes nigrum), and the emulsifier caprylyl/capryl glucoside.

While the skin care agents known from the prior art already satisfy high requirements in terms of the cosmetic effectiveness thereof, a need continues to exist for skin care cosmetics having improved cosmetic effectiveness and a long shelf life.

It is therefore desirable to provide skin care cosmetics that include cosmetically active plant extracts, are present in storage-stable form, are both chemically and physically stable, and distinguish themselves by good cosmetic properties, and in particular by low viscidity and good skin tolerability.

Furthermore, other desirable features and characteristics of the present invention will become apparent from the subsequent detailed description of the invention and the appended claims, taken in conjunction with this background of the invention.

### BRIEF SUMMARY OF THE INVENTION

An aqueous cosmetic composition includes at least one alkyl glucoside including a linear or branched alkyl or alkenyl group having 14 to 30 carbon atoms and a mono-, di- or triglucoside group; at least one extract of a plant species from the aralia family (Araliaceae); at least one extract of a plant species from the pine family (Pinaceae); at least one extract of a plant species from the flowering plants family (Theaceae); and at least one extract of a plant species from the gooseberry family (Grossulariaceae).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has now been found that skin care cosmetics that, in addition to specific plant extracts, furthermore include an alkyl glucoside having a long-chain alkyl chain, achieve the above-described objects.

The present invention provides:

1. An aqueous cosmetic composition, comprising:

a) at least one alkyl glucoside including a linear or branched alkyl or alkenyl group having 14 to 30 carbon atoms and a mono-, di- or triglucoside group;

b) at least one extract of a plant species from the aralia family (Araliaceae);

c) at least one extract of a plant species from the pine family (Pinaceae);

d) at least one extract of a plant species from the gooseberry family (Grossulariaceae); and

e) at least one extract of a plant species from the flowering plants family (Theaceae).

2. The cosmetic composition according to item 1, wherein the alkyl glucoside includes a linear alkyl group having 16 to 18 carbon atoms and a monoglucoside group.
3. The cosmetic composition according to any one of the preceding items, wherein the alkyl glucoside, based on the total weight of the composition, is present in amounts of 0.1 to 4.0 wt. %, preferably of 0.2 to 3.0 wt. %, and in particular of 0.5 to 2.0 wt. %.
4. The cosmetic composition according to any one of the preceding items, wherein the extract of a plant species from the aralia family (Araliaceae) is selected from the extracts of plants from the Panax genus, preferably from extracts of the ginseng species (Panax ginseng), and in particular from root extracts of the ginseng species (Panax ginseng).
5. The cosmetic composition according to any one of the preceding items, wherein the extract of a plant species from the aralia family (Araliaceae), based on the total weight of the composition, is present in amounts of 0.3 to 1.2 wt. %, preferably of 0.35 to 0.9 wt. %, and in particular of 0.4 to 0.5 wt. %.
6. The cosmetic composition according to any one of the preceding items, wherein the extract of a plant species from the pine family (Pinaceae) is selected from the extracts of plants from the pine genus (Pinus), preferably from extracts of the Scots pine species (Pinus sylvestris), and in particular from bark extracts of the Scots pine species (Pinus sylvestris).
7. The cosmetic composition according to any one of the preceding items, wherein the extract of a plant species from the pine family (Pinaceae), based on the total weight of the composition, is present in amounts of 0.01 to 0.1 wt. %, preferably of 0.015 to 0.05 wt. %, and in particular of 0.02 to 0.03 wt. %.
8. The cosmetic composition according to any one of the preceding items, wherein the extract of a plant species from the gooseberry family (Grossulariaceae) is selected from the extracts of plants from the currant genus (Ribes), preferably from extracts of the black currant species (Ribes nigrum), and in particular from leaf extracts of the black currant species (Ribes nigrum).
9. The cosmetic composition according to any one of the preceding items, wherein the extract of a plant species from the gooseberry family (Grossulariaceae), based on the total weight of the composition, is present in amounts of 0.01 to 0.1 wt. %, preferably of 0.015 to 0.05 wt. %, and in particular of 0.02 to 0.03 wt. %.
10. The cosmetic composition according to any one of the preceding items, wherein the extract of a plant species from the flowering plants family (Theaceae) is selected from the extracts of plants from the camellia genus (Camellia), preferably from extracts of the tea species (Camellia sinensis), and in particular from leaf extracts of the tea species (Camellia sinensis).
11. The cosmetic composition according to any one of the preceding items, wherein the extract of a plant species from the flowering plants family (Theaceae), based on the total weight of the composition, is present in amounts of 0.2 to 1.2 wt. %, preferably of 0.30 to 0.9 wt. %, and in particular of 0.4 to 0.6 wt. %.
12. The cosmetic composition according to any one of the preceding items, wherein the composition furthermore includes

f) at least one anionic oil-in-water emulsifier, selected from the salts of C₁₂₋₂₀ alkyl phosphate, and in particular from the salts of cetyl phosphate;

g) at least one C₁₄₋₂₀ monoacylglyceride or diacylglyceride, preferably at least one C₁₆₋₁₈ monoacylglyceride or diacylglyceride, and particularly preferably selected from hydrogenated palm glycerides.

13. The cosmetic composition according to item 12, wherein the salts of C₁₂₋₂₀ alkyl phosphate, and in particular the salts of cetyl phosphate, based on the total weight of the composition, are present in amounts of 0.1 to 5.0 wt. %, preferably 0.1 to 3.5 wt. %, and in particular 0.1 to 2.0 wt. %.
14. The cosmetic composition according to item 12 or 13, wherein the C₁₄₋₂₀ monoacylglycerides or diacylglycerides, preferably the C₁₆₋₁₈ mono- or diacylglycerides, and particularly preferably the hydrogenated palm glycerides, based on the total weight of the composition, are present in amounts of 0.05 to 3.0 wt. %, preferably 0.05 to 1.5 wt. %, and in particular 0.05 to 1.0 wt. %.
15. The cosmetic composition according to any one of items 12 to 14, wherein the salts of C₁₂₋₂₀ alkyl phosphate and the C₁₄₋₂₀ monoacylglycerides and C₁₄₋₂₀ diacylglycerides are present in a weight ratio of 3:1 to 1:1.5, and particularly preferably of 2.5:1 to 1:1.
16. The cosmetic composition according to any one of items 12 to 15, wherein the salts of cetyl phosphate and the hydrogenated palm glycerides are present in a weight ratio of 3:1 to 1:1.5, and particularly preferably of 2.5:1 to 1:1.
17. The cosmetic composition according to any one of the preceding items, wherein the composition, based on the total weight thereof, includes 60 to 95 wt. %, preferably 70 to 90 wt. %, and in particular 75 to 85 wt. % water.
18. The cosmetic composition according to any one of the preceding items, wherein the composition, based on the total weight thereof, includes 4.0 to 18 wt. %, preferably 6.0 to 15 wt. %, and in particular of 8.0 to 12 wt. % alcohol, preferably alcohol from the group consisting of glycerol, 1,6-hexanediol and dipropylene glycol.
19. The cosmetic composition according to any one of the preceding items, wherein the composition, based on the total weight thereof, includes 0.1 to 4.0 wt. %, preferably 0.2 to 3.0 wt. %, and in particular of 0.5 to 2.0 wt. % of at least one starch compound, and in particular of an aluminum starch octenylsuccinate.
20. A method for cosmetic skin care, in which a cosmetic composition according to any one of items 1 to 19 is applied to the skin, and in particular to the facial skin.

An exemplary embodiment of the present invention is an aqueous cosmetic composition that includes:

a) at least one alkyl glucoside including a linear or branched alkyl or alkenyl group having 14 to 30 carbon atoms and a mono-, di- or triglucoside group;

b) at least one extract of a plant species from the aralia family (Araliaceae);

c) at least one extract of a plant species from the pine family (Pinaceae);

d) at least one extract of a plant species from the flowering plants family (Theaceae); and

e) at least one extract of a plant species from the gooseberry family (Grossulariaceae).

The cosmetic compositions according to the invention are aqueous, which is to say include an aqueous carrier. The composition according to the invention is preferably viscous-liquid. Preferred cosmetic compositions, based on the total weight thereof, are characterized by a water content of 60 to 95 wt. %, preferably 70 to 90 wt. %, and in particular 75 to 85 wt. %.

In addition to water, the compositions, based on the total weight thereof, can furthermore include 4.0 to 18 wt. %, preferably 6.0 to 15 wt. %, and in particular 8.0 to 12 wt. % of at least one alcohol. Preferred alcohols are glycol, 1,2-propylene glycol, 1,3-propylene glycol, 2-methyl-1,3-propanediol, glycerol, 1,2-butylene glycol, 1,3-butylene glycol, 1,4-butylene glycol, pentylene glycol, 1,2-hexanediol, 1,6-hexanediol, 1,2,6-hexanetriol, 1,2-octanediol, 1,8-octanediol, dipropylene glycol, tripropylene glycol, diglycerol, triglycerol, erythritol, sorbitol, polyethylene glycol, and mixtures of the aforementioned substances.

Particularly preferably, the alcohol that the compositions include is 1,2-propylene glycol, glycerol, 1,3-butylene glycol, 1,6-hexanediol, diglycerol, triglycerol, dipropylene glycol and/or tripropylene glycol, and in particular glycerol, 1,6-hexanediol and dipropylene glycol.

As a first essential component, the compositions according to the invention include an alkyl glucoside including a linear or branched alkyl or alkenyl group having 14 to 30 carbon atoms and a mono-, di- or triglucoside group, preferably a monoglycoside group.

Suitable alkyl glucosides correspond to the general formula RO—(Z)ₓ, wherein R denotes a linear or branched C₁₄ to C₃₀ alkyl or alkenyl group, and preferably a linear C₁₄ to C₂₀ alkyl group, Z denotes a sugar residue, such as glucose, and x, serving as an indicator of the number of sugar units, denotes a value of 1, 2 or 3.

Given their cosmetic properties and high stability, preferred compositions according to the invention are those in which the alkyl glucoside includes a linear alkyl group having 16 to 18 carbon atoms and a monoglucoside group.

Examples of alkyl glucosides are compounds having the INCI names Decyl Glucoside (commercial product Plantaren 2000), Lauryl Glucoside (commercial product Plantacare 1200), Coco Glucoside (commercial product Plantacare 818), Capryl/Caprylyl Glucoside (commercial product Oramix CG 110). A preferred alkyl glucoside according to the invention bears the INCI name Cetearyl Glucoside (commercial product Tego Care CG 90).

The percentage by weight of the alkyl glucoside in the total weight of the composition is preferably 0.1 to 4.0 wt. %, especially 0.2 to 3.0 wt. %, and in particular 0.5 to 2.0 wt. %.

Further essential components that the compositions include are four plant extracts.

The first of these extracts is an extract of a plant species from the aralia family (Araliaceae). The aralia family is a plant family in the umbellifer order (Apiales) and widely common worldwide, predominantly in the tropics.

Within the aralia family, extracts of plants from the Panax genus, and preferably extracts of the ginseng species (Panax ginseng), are preferred due to the cosmetic effectiveness and chemical and physical stability thereof. Root extracts of ginseng (Panax ginseng) are particularly preferred.

The percentage by weight of the aralia extract, based the total weight of the composition, is preferably 0.3 to 1.2 wt. %, especially 0.35 to 0.9 wt. %, and in particular 0.4 to 0.5 wt. %.

A further extract characterizing the invention is an extract of a plant species from the pine family (Pinaceae). The pine family forms a plant family in the conifer order. With the exception of a few deciduous larches, they are evergreen trees and common primarily in the northern hemisphere.

Given the cosmetic effectiveness and chemical and physical stability, preferred extracts according to the invention come from plants of the pine genus (Pinus), and preferably plants of the Scots pine species (Pinus sylvestris). Bark extracts of the Scots pine species (Pinus sylvestris) are particularly preferred.

Based on the total weight of the cosmetic composition, the percentage by weight of the extract of a plant species from the pine family (Pinaceae) is especially 0.01 to 0.1 wt. %, preferably 0.015 to 0.05 wt. %, and in particular 0.02 to 0.03 wt. %.

The third extract characterizing the cosmetic composition is the extract of a plant species from the gooseberry family (Grossulariaceae), wherein extracts of plants from the currant genus (Ribes), and in particular of plants from the black currant species (Ribes nigrum), are preferred due to the cosmetic effectiveness and chemical and physical stability thereof. Leaf extracts of the black currant species (Ribes nigrum) are particularly preferred.

Cosmetic compositions in which the extract of a plant species from the gooseberry family (Grossulariaceae), based on the total weight of the composition, accounts for 0.01 to 0.1 wt. %, especially of 0.015 to 0.05 wt. %, and in particular of 0.02 to 0.03 wt. %, are preferred.

The fourth extract used in the compositions is the extract of a plant species from the flowering plants family (Theaceae). The flowering plants family belongs to the heather family and can be found in the tropics and subtropics. Extracts of plants from the camellia genus (Camellia), and preferably extracts of the tea species (Camellia sinensis) are preferred due to the cosmetic effectiveness and chemical and physical stability thereof. Leaf extracts of the tea species (Camellia sinensis) are particularly preferred. The percentage by weight of the extract of the plant species from the flowing plants family (Theaceae) in the total weight of the composition, is especially 0.2 to 1.2 wt. %, preferably 0.30 to 0.9 wt. %, and in particular 0.4 to 0.6 wt. %.

In summary, particularly preferred aqueous cosmetic compositions include:

a) 0.1 to 4.0 wt. % of at least one alkyl glucoside comprising a linear alkyl group having 16 to 18 carbon atoms and a monoglucoside group;

b) at least one root extract of the ginseng species (Panax ginseng);

c) at least one bark extract of the Scots pine species (Pinus sylvestris);

d) at least one leaf extract of the black currant species (Ribes nigrum); and

e) at least one leaf extract of the tea species (Camellia sinensis).

In addition to the above-described ingredients, the cosmetic compositions may include further active and auxiliary substances.

To enhance the cosmetic effectiveness and the chemical and physical stability, preferred cosmetic compositions, for example, furthermore include:

a) at least one anionic oil-in-water emulsifier, selected from the salts of C₁₂₋₂₀ alkyl phosphate, and in particular from the salts of cetyl phosphate;

b) at least one C₁₄₋₂₀ monoacylglyceride or diacylglyceride, preferably at least one C₁₆₋₁₈ monoacylglyceride or diacylglyceride, and particularly preferably selected from hydrogenated palm glycerides.

For the cosmetic properties, but in particular for the shelf life of the cosmetic compositions, it has proven advantageous if the salts of C₁₂₋₂₀ alkyl phosphate, and in particular the salts of cetyl phosphate, based on the total weight of the composition, are present in amounts of 0.1 to 5.0 wt. %, preferably 0.1 to 3.5 wt. %, and in particular 0.1 to 2.0 wt. %.

The salts of C₁₂₋₂₀ alkyl phosphates may be present, for example, as alkali, alkaline earth, ammonium, alkyl ammonium, alkanolamine or glucammonium salt. Preferred are the corresponding sodium, potassium, alkanolamine, trialkylammonium, triethanolamine, 2-amino-1-butanol, 2-amino-2-methyl-1-propanol, 2-amino-2-methyl-1,3-propanediol, 2-amino-2-ethyl-1,3-propanediol or tris-(hydroxymethyl)aminomethane salts, and salts of the basic amino acids ornithine, lysine, arginine and/or histidine. The potassium salts of said C₁₂₋₂₀ alkyl phosphates are particularly preferred.

The group of the salts of C₁₂₋₂₀ alkyl phosphates includes the salts of phosphoric acid C₁₂₋₂₀ monoalkyl esters and the salts of phosphoric acid C₁₂₋₂₀ dialkyl esters.

Preferred salts of C₁₂₋₂₀ alkyl phosphates according to the invention are selected from the monoesters of phosphoric acid with lauryl alcohol, tridecyl alcohol, isotridecyl alcohol, myristyl alcohol, pentadecyl alcohol, cetyl alcohol, palmityl alcohol, isocetyl alcohol, isostearyl alcohol, stearyl alcohol, oleyl alcohol, elaidyl alcohol, linoleyl alcohol, linolenyl alcohol, nonadecyl alcohol, arachyl alcohol, gadoleyl alcohol or arachidyl alcohol, which are present as alkali, alkaline earth, ammonium, alkyl ammonium, alkanolamine or glucammonium salt, preferably the corresponding sodium, potassium, alkanolamine, trialkylammonium, triethanolamine, 2-amino-1-butanol, 2-amino-2-methyl-1-propanol, 2-amino-2-methyl-1,3-propanediol, 2-amino-2-ethyl-1,3-propanediol or tris-(hydroxymethyl)aminomethane salts, and salts of the basic amino acids ornithine, lysine, arginine and/or histidine. The potassium salts of the aforementioned phosphoric acid monoesters are preferred. Dipotassium monocetyl phosphate is particularly preferred.

Further preferred salts of C₁₂₋₂₀ alkyl phosphates according to the invention are selected from the diesters of phosphoric acid with lauryl alcohol, tridecyl alcohol, isotridecyl alcohol, myristyl alcohol, pentadecyl alcohol, cetyl alcohol, palmityl alcohol, isocetyl alcohol, isostearyl alcohol, stearyl alcohol, oleyl alcohol, elaidyl alcohol, linoleyl alcohol, linolenyl alcohol, nonadecyl alcohol, arachyl alcohol, gadoleyl alcohol or arachidyl alcohol, which are present as alkali, alkaline earth, ammonium, alkyl ammonium, alkanolamine or glucammonium salt, preferably the corresponding sodium, potassium, alkanolamine, trialkylammonium, triethanolamine, 2-amino-1-butanol, 2-amino-2-methyl-1-propanol, 2-amino-2-methyl-1,3-propanediol, 2-amino-2-ethyl-1,3-propanediol or tris-(hydroxymethyl)aminomethane salts, and salts of the basic amino acids ornithine, lysine, arginine and/or histidine. The potassium salts of the aforementioned phosphoric acid diesters are preferred. Potassium dicetyl phosphate is particularly preferred.

Mixtures of mono C₁₂₋₂₀ alkyl phosphates and di C₁₂₋₂₀ alkyl phosphates are particularly preferred. Mixtures of dipotassium monocetyl phosphate and potassium dicetyl phosphate are exceptionally preferred.

Preferred compositions include at least one C₁₄₋₂₀ monoacylglyceride or diacylglyceride as a further component. The content of the C₁₄₋₂₀ monoacylglyceride or diacylglyceride, preferably of the C₁₆₋₁₈ monoacylglyceride or diacylglyceride, and particularly preferably of the hydrogenated palm glyceride in the total weight of the composition is especially 0.05 to 3.0 wt. %, preferably 0.05 to 1.5 wt. %, and in particular 0.05 to 1.0 wt. %. Corresponding compositions have a particularly good chemical and physical stability and good cosmetic properties.

Preferred C₁₄₋₂₀ monoacylglycerides or diacylglycerides according to the invention are selected from monomyristoyl glyceride, monopalmitoyl glyceride, monostearoyl glyceride, monoarachinoyl glyceride, dimyristoyl glyceride, dipalmitoyl glyceride, distearoyl glyceride, diarachinoyl glyceride, and mixtures of two or more compounds thereof. Further preferred C₁₄₋₂₀ monoacylglycerides or diacylglycerides according to the invention are glycerides of hydrogenated, and preferably fully hydrogenated, fatty acids of natural oils. Particularly preferred C₁₆₋₁₈ monoacylglycerides or diacylglycerides are selected from the group of hydrogenated palm glycerides (INCI: Hydrogenated Palm Glycerides).

Aside from the percentage by weight of cetyl phosphate and diacylglyceride, the properties of the cosmetic compositions can furthermore also advantageously be influenced by the weight ratios of the two above-described components. The cosmetic composition in which the salts of C₁₂₋₂₀ alkyl phosphate and the C₁₄₋₂₀ monoacylglycerides and C₁₄₋₂₀ diacylglycerides are present in a weight ratio of 3:1 to 1:1.5, and preferably of 2.5:1 to 1:1, are characterized by a long shelf life. Cosmetic compositions in which the salts of cetyl phosphate and the hydrogenated palm glycerides are present in a weight ratio of 3:1 to 1:1.5, and particularly preferably of 2.5:1 to 1:1, are particularly preferred.

The compositions preferably furthermore include a starch compound, and preferably a modified starch compound. One example of such a modified starch compound is the aluminum starch octenylsuccinate, an aluminum salt of corn starch modified with 2-octylsuccinic acid (commercial product: DryFlo Plus or DryFlo Pure).

The cosmetic composition preferably includes the starch compound, and in particular the aluminum starch octenylsuccinate, based on the total weight thereof, in amounts of 0.1 to 4.0 wt. %, preferably 0.2 to 3.0 wt. %, and in particular 0.5 to 2.0 wt. %.

Tabular Overview

The make-up of several preferred cosmetic compositions can be derived from the following tables (information in percent by weight refers to the total weight of the cosmetic composition, unless indicated otherwise).

| | Formula | Formula | Formula | Formula |
|---|---|---|---|---|
| | 1 | 2 | 3 | 4 |
| C₁₄₋₃₀ alkyl | 0.1 to 4.0 | 0.2 to 3.0 | 0.2 to 3.0 | 0.5 to 2.0 |
| glucoside | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 5 | 6 | 7 | 8 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 11 | 12 | 13 | 14 |
| C₁₄₋₃₀ alkyl | 0.1 to 4.0 | 0.2 to 3.0 | 0.2 to 3.0 | 0.5 to 2.0 |
| glucoside | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 15 | 16 | 17 | 18 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 21 | 22 | 23 | 24 |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 25 | 26 | 27 | 28 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 31 | 32 | 33 | 34 |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 35 | 36 | 37 | 38 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 41 | 42 | 43 | 44 |
| C₁₄₋₃₀ alkyl | 0.1 to 4.0 | 0.2 to 3.0 | 0.2 to 3.0 | 0.5 to 2.0 |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 45 | 46 | 47 | 48 |
| C₁₄₋₃₀ alkyl | 0.1 to 4.0 | 0.2 to 3.0 | 0.2 to 3.0 | 0.5 to 2.0 |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 51 | 52 | 53 | 54 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 55 | 56 | 57 | 58 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 61 | 62 | 63 | 64 |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 65 | 66 | 67 | 68 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 71 | 72 | 73 | 74 |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 75 | 76 | 77 | 78 |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Misc | to make | to make | to make | to make |
| | 100 | 100 | 100 | 100 |
| | Formula | Formula | Formula | Formula |
| | 1a | 2a | 3a | 4a |
| C₁₄₋₃₀ alkyl | 0.1 to 4.0 | 0.2 to 3.0 | 0.2 to 3.0 | 0.5 to 2.0 |
| glucoside | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 5a | 6a | 7a | 8a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 11a | 12a | 13a | 14a |
| C₁₄₋₃₀ alkyl | 0.1 to 4.0 | 0.2 to 3.0 | 0.2 to 3.0 | 0.5 to 2.0 |
| glucoside | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 15a | 16a | 17a | 18a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 21a | 22a | 23a | 24a |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 25a | 26a | 27a | 28a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 31a | 32a | 33a | 34a |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 35a | 36a | 37a | 38a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 41a | 42a | 43a | 44a |
| C₁₄₋₃₀ alkyl | 0.1 to 4.0 | 0.2 to 3.0 | 0.2 to 3.0 | 0.5 to 2.0 |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 45a | 46a | 47a | 48a |
| C₁₄₋₃₀ alkyl | 0.1 to 4.0 | 0.2 to 3.0 | 0.2 to 3.0 | 0.5 to 2.0 |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 51a | 52a | 53a | 54a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 55a | 56a | 57a | 58a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 61a | 62a | 63a | 64a |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 65a | 66a | 67a | 68a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Araliaceae | present | 0.3 to 1.2 | 0.35 to 0.9 | 0.4 to 0.5 |
| extract | | | | |
| Pinaceae | present | 0.01 to 0.1 | 0.015 to 0.05 | 0.02 to 0.03 |
| extract | | | | |
| Grossular- | present | 0.01 to 0.1 | 0.015 to 0.05 | 0.02 to 0.03 |
| iaceae | | | | |
| extract | | | | |
| Theaceae | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 71a | 72a | 73a | 74a |
| C₁₄₋₃₀ alkyl | 0.1 to 4.0 | 0.2 to 3.0 | 0.2 to 3.0 | 0.5 to 2.0 |
| glucoside | | | | |
| C₁₂₋₂₀ alkyl | 0.1 to 5.0 | 0.1 to 3.5 | 0.1 to 3.5 | 0.1 to 2.0 |
| phosphate | | | | |
| salt | | | | |
| C₁₄₋₂₀ | 0.05 to 3.0 | 0.05 to 3.0 | 0.05 to 1.5 | 0.05 to 1.0 |
| mono- | | | | |
| acylglyceride | | | | |
| or | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Camellia | present | 0.2 to 1.2 | 0.3 to 0.9 | 0.4 to 0.6 |
| sinensis | | | | |
| leaf extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |
| | Formula | Formula | Formula | Formula |
| | 75a | 76a | 77a | 78a |
| C₁₆₋₁₈ alkyl | 0.1 to 4.0 | 0.2 to 3.0 | 0.2 to 3.0 | 0.5 to 2.0 |
| mono- | | | | |
| glucoside | | | | |
| Cetyl | 0.1 to 5.0 | 0.1 to 3.5 | 0.1 to 3.5 | 0.1 to 2.0 |
| phosphate | | | | |
| C₁₆₋₁₈ | 0.05 to 3.0 | 0.05 to 3.0 | 0.05 to 1.5 | 0.05 to 1.0 |
| mono- | | | | |
| acylglyceride | | | | |
| and | | | | |
| diacyl- | | | | |
| glyceride | | | | |
| Aluminum | 0.1 to 4.0 | 0.2 to 3.0 | 0.2 to 3.0 | 0.5 to 2.0 |
| starch | | | | |
| octenyl- | | | | |
| succinate | | | | |
| Panax | present | 0.3 to 1.2 | 0.35 to 0.9 | 0.4 to 0.5 |
| ginseng | | | | |
| root extract | | | | |
| Pinus | present | 0.01 to 0.1 | 0.015 to 0.05 | 0.02 to 0.03 |
| sylvestris | | | | |
| bark extract | | | | |
| Ribes nigrum | present | 0.01 to 0.1 | 0.015 to 0.05 | 0.02 to 0.03 |
| leaf extract | | | | |
| Alcohol ¹⁾ | present | 0.2 to 1.2 | 0.3 to 0.9 | 0.4 to 0.6 |
| extract | | | | |
| Water | 60 to 95 | 60 to 95 | 70 to 90 | 75 to 85 |
| Alcohol ¹⁾ | 4.0 to 18 | 4.0 to 18 | 6.0 to 15 | 8.0 to 12 |
| Misc | to make | to make | to make | to make |
| | 100 | 100 | 100 | 100 |

¹⁾ preferably alcohol from the group consisting of glycerol and 1,6-hexanediol

A third subject matter of the present invention is finally a method for cosmetic skin care, in which a cosmetic composition according to the invention is applied to the skin, and in particular to the facial skin.

What was said above with respect to the cosmetic agents according to the invention applies, mutatis mutandis, to the cosmetic method according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An aqueous cosmetic composition, including:
a) at least one alkyl glucoside including a linear or branched alkyl or alkenyl group having 14 to 30 carbon atoms and a mono-, di- or triglucoside group;
b) at least one extract of a plant species from the aralia family (Araliaceae);
c) at least one extract of a plant species from the pine family (Pinaceae);
d) at least one extract of a plant species from the flowering plants family (Theaceae); and
e) at least one extract of a plant species from the gooseberry family (Grossulariaceae).

2. The cosmetic composition according to claim 1, wherein the alkyl glucoside includes a linear alkyl group having 16 to 18 carbon atoms and a monoglucoside group.

3. The cosmetic composition according to claim 1, wherein the alkyl glucoside, based on the total weight of the composition, is present in an amount of 0.1 to 4.0 wt. %.

4. The cosmetic composition according claim 1, wherein the extract of a plant species from the aralia family (Araliaceae) is selected from the extracts of plants from the Panax genus.

5. The cosmetic composition according to claim 1, wherein the extract of a plant species from the pine family (Pinaceae) is selected from the extracts of plants from the pine genus (Pinus).

6. The cosmetic composition according to claim 1, wherein the extract of a plant species from the gooseberry family (Grossulariaceae) is selected from the extracts of plants from the currant genus (Ribes).

7. The cosmetic composition according to claim 1, wherein the extract of a plant species from the flowering plants family (Theaceae) is selected from the extracts of plants from the camellia genus (Camellia).

8. The cosmetic composition according to claim 1, wherein the composition furthermore includes:
f) at least one anionic oil-in-water emulsifier, selected from the salts of C₁₂₋₂₀ alkyl phosphate; and
g) at least one C₁₄₋₂₀ monoacylglyceride or diacylglyceride.

9. The cosmetic composition according to claim 1, wherein the composition, based on the total weight thereof, includes 60 to 95 wt. % water.

10. A method for cosmetic skin care, in which a cosmetic composition according to claim 1 is applied to the skin.
